(19) United States
(12) Patent Application Publication
(10) Pub. No.: US 2019/0184012 A1
(43) Pub. Date: Jun. 20, 2019

(54) NANOBODY BIOMEDICINE TRANSDERMAL ADMINISTRATION FORMULATION SYSTEM AND PREPARATION METHOD AND USE THEREOF

(71) Applicant: Nanolattix Biotechnology Co. Ltd., Taiyuan, Shanxi (CN)

(72) Inventors: Zhican Qu, Taiyuan, Shanxi (CN); Shaoping LI, Taiyuan, Shanxi (CN)

(21) Appl. No.: 16/068,668

(22) PCT Filed: Sep. 8, 2017
(86) PCT No.: PCT/CN2017/101028

§ 371 Date: Jul. 7, 2018

(30) Foreign Application Priority Data
Sep. 3, 2016 (CN) 201610804324.X

(51) Int. Cl. A61K 39/395; A61K 9/06; A61K 47/32; A61K 47/36; A61K 47/18; A61K 47/10; A61K 47/24; A61K 47/42; A61K 47/38

(52) U.S. Cl. CPC A61K 39/395; A61K 9/06; A61K 47/32; A61K 47/36; A61K 2039/54; A61K 47/10; A61K 47/24; A61K 47/42; A61K 47/38; A61K 47/183

(57) ABSTRACT

The invention discloses a transdermal drug delivery scheme for preparing a specific nanobody biopharmaceutical through penetrating skin tissue, and its preparation method and application. Due to biological characteristics such as unstable activity of traditional antibodies or protein macromolecules, the administration of biopharmaceuticals is basically limited to injections. Nanobodies are the smallest units currently known to bind target antigens. The three-dimensional structure of nanobodies determines the relative stability of their structure and biological activity and their biological properties. Nanobodies have the characteristics of high temperature resistance, activity in a certain acid-base environment, good water solubility, and strong tissue penetration. Therefore, by optimizing the formulation of the carrier, the stability of the nanobody and the penetration of the skin tissue can be maintained and increased, so as to achieve a transdermal delivery formulation of the nanobody biopharmaceutical. The transdermal delivery of nanobody can be used for the specific nanobody to play a role in the local application area, and can also be used for the nanobody to penetrate the skin and enter the blood circulation to reach the site of a predetermined lesion. Nanobodies in the transdermal delivery system include specific nanobodies for different lesion targets, such as, but not limited to, dermatological targets, inflammation and rheumatism targets, cancer targets, viral bacteria targets, cardiovascular disease targets, diabetes targets, Alzheimer's disease targets, brain tumor targets. The carrier for transdermal delivery of the nanobody has the advantages of stabilizing the structure and efficacy of the nanobody, releasing the drug via the skin, and having good transdermal effect; the comfort of skin application is high; and the skin irritation and allergic reaction characteristics are basically absent. The carrier system components include, but not limited to, soluble polymer macromolecular carbohydrate matrix, polyethylene glycol (PEG), dextran, polyamino acid, optimized formulation, stirred mixture. The invention discloses a preparation method and application of the transdermal drug delivery system. Transdermal administration is a safe and effective method of administration. Less content of proteolytic enzymes in the skin tissue is conducive to maintaining the stability of such biological drugs. The nanobody biomedical transdermal drug delivery preparation system has the advantages of being non-toxic, stable, controllable, convenient for administration, and easy to operate, etc., and opens up new dosage forms of nanobody biopharmaceuticals, and its application prospect is very broad.

Nanoparticle biologic drug transdermal drug delivery preparation system and preparation method and application

### TECHNICAL FIELD

The present invention relates to the field of medical technology, and in particular to a method system for in vitro administration of a specific nanobody biopharmaceutical through the skin, and a method for preparing a nanobody-transdermally-administered agent and application of these products to a lesion.

### BACKGROUND TECHNIQUE

Belgian scientists reported for the first time in 1993 that half of the antibodies in the blood of camels have no light chains, and these heavy chain antibodies (HCAbs) that deplete the light chain can react with antigens like normal antibodies. The targets are tightly bound, and in addition they do not stick to each other like scFvs, or even aggregate into blocks. In short, nanobodies are characterized by their high affinity and high specificity, while their immunogenicity (although they are non-human, but their immunogenicity is very low) and their toxicity are very low and they are not as easy to adhere as scFv.

This kind of antibody contains only a variable domain of heavy chain of HCAb (VHH) and two conventional CH2 and CH3 regions, and more importantly, the VHH region cloned and expressed independently has very good structural stability and antigen binding activity. VHH is currently known as the smallest unit that binds the target antigen, so VHH is also called a Nanobody.

Compared with the human antibody VH, the CDR3 is longer, can form a convex ring structure, can penetrate into the antigen inside and combine with the antigen better, and thus has a higher affinity. In addition, the hydrophobic residue of FR2 of VHH is replaced with a hydrophilic residue, which is more water soluble and less likely to form aggregates.

Nanobodies are the smallest units currently known to bind target antigens. VHH crystal is 2.5 nm, 4 nm in length, and the molecular weight is only 15 KDa. It's molecular structure is relatively stable, can withstand high temperature and maintain its activity in extremely harsh environments. Studies have confirmed that VHH can retain 80% of biological activity after being left at 37° C. for 1 week, indicating that the Nanobody is quite stable at room temperature, which makes it easier to store and transport than conventional antibodies. Nanobodies have strong and rapid tissue penetrating ability, which facilitates their entry into dense tissues such as solid tumors, and can effectively penetrate the blood-brain barrier, providing a new method for brain administration.

At the same time, the nanobody has a reversible unfolding ability, and the test shows that the nanobody still maintains a high activity after being treated at a high temperature of 90° C. and can still regain the antigen-binding ability. All conventional antibodies lost their activity after treatment at 90° C. and irreversible polymerization occurred. Under severe conditions, such as chaotropic agents, presence of proteases, and extreme pH denaturation, normal antibodies can fail or break down, while nanobodies still have a high degree of stability.

In addition, nano-antibodies also exhibit the characteristics of being less prone to denaturation or variability after denaturation under the conditions of strong denaturants. Utilizing the characteristics and advantages of nanobodies, by optimizing the formulation of the carrier, maintaining and increasing the stability of the nanobody and the penetration of the skin tissue, a transdermal in vitro dosage form of the nanobody can be realized.

Compared with conventional antibodies, nanobodies are readily available (immunization, B-lymphocyte separation, screening by antibody library display technology), and good stability (internal folds contain multiple disulfide bonds, making their structures very good stability, can be placed at room temperature), high solubility (not easy to cluster together like scFv, VHH good hydrophilicity, good water solubility, improved utilization as a drug), good absorption (because of high solubility), so nanobody has the advantage of high absorption rate. Moreover, nanobody expression is easy (unlike traditional antibodies which must be expressed in mammalian cells. difficulties, low yield, high cost, VHH can be highly expressed in prokaryotic cells, some researchers can increase the output to 2.5 g/L), humanization is simple (homology with human heavy chain is 80%-90%, humanization has been successful). At the same time, nanobodies easily pass through the biomembrane system and couple with other molecules easily.

Of course, the half-life of nanobodies is not very long, and it is necessary to prolong the half-life of nanobodies in antibody drug carriers and human bodies. The half-life of nanobodies in antibody drug carriers and human body will be greatly extended by means of adaptations such as fusion of nanobodies and albumin, Fc fusion, and PEGylation.

With the continuous development of bioengineering technology, a large number of nanobody drugs continue to emerge. At present, its main clinical dosage forms are injections and oral agents. Due to the small molecular weight of nanobody drugs, the stability in vivo is poor. When oral administration, it is susceptible to degradation by enzymes in the gastrointestinal tract and the first-pass effect of the liver enzyme system. Therefore, in order to achieve an effective drug therapeutic concentration, the patient needs to be administered repeatedly over a long period of time.

A transdermal or in vitro drug delivery system refers to a controlled release drug system transdermal drug delivery system that facilitates the passage of a therapeutic amount of drug through the skin into the systemic circulation. Poor absorption of gastrointestinal tract due to gastrointestinal pH, enzymes, food, and other drug interactions can be avoided; first-pass effects can be avoided; inconvenience caused by injections can also be avoided; and efficacy after a single administration can be prolonged. Through drug storage and controlled release characteristics to control the treatment time of drugs with short half-life; also can be torn off at any time to stop the drug; and can be used in emergency patients without response, unaware of coma patients. The transdermal drug delivery system is the focus of modern pharmacy research.

The transdermal drug delivery system of nanobodies is undoubtedly the most innovative innovation, and it is suitable for the characteristics of nanobodies. Nanobody is a safe and effective method of administration by percutaneous or in vitro administration, and the content of proteolytic enzymes in the skin tissue is less, which is conducive to maintaining the stability of such drugs, but due to the small molecular weight of the nanobody, it is easy to pass through biomembrane systems and achieve the desired transdermal absorption. Therefore, we have pioneered an effective transdermal delivery system for novel nanobodies that are non-toxic, cost-effective, and easy to use.

There is no relevant reports about the transdermal or in vitro administration of nanobody formulation in the world, so it is belong to the international initiative.

### SUMMARY OF THE INVENTION

The technical problem to be solved by the present invention is to provide a percutaneous or in vitro drug delivery system for a nanobody, which has the advantages of being non-toxic, low cost, and convenient to use, and is particularly suitable for transdermal delivery of nanobody drugs and other drugs.

In addition, there is also a need to provide a preparation method and application for implementing the above-described transdermal or in vitro drug delivery system.

The following examples illustrate the understanding of the embodiments of the present invention. However, it should be noted that the followings are only examples or illustrations of application of the principle of the present invention. Technicians in the field can devise many variations and alternative compositions, methods, and systems without departing from the spirit and scope of the present invention. The appended claims are therefore intended to cover these changes and arrangements. Although the present invention is described below in detail by way of example, the following embodiments provide only one of the details of an embodiment of the present invention. Numerous variations and modifications may be made without departing from the concept of the core content of the present invention: transdermal or in vitro administration system of nanobody drug, and these are all within the protection scope of the present invention.

The purpose of the embodiments of the present invention is to provide a method for preparing a transdermal sustained-release drug delivery system based on a water-soluble polymer material and a preparation application.

Implementation of one of the technical solutions of the present invention: A transdermal drug delivery system using a water-soluble polymer protein material as a base, and the basic formulation of the drug delivery system is prepared from the following auxiliary materials in parts by weight: 18 parts of water-soluble high molecular weight biomatrix, 10 parts of polyhydroxyl compound, 5 parts of polyvinyl alcohol (PEG), 10 parts of levulose, 10 parts of polyamino acids, 10 parts of glycerol, 5 parts of phospholipids, 2.5 parts of gelatin, 1 part of carboxymethyl sodium cellulose, mix with water.

Another object of embodiments of the present invention is to provide a method for preparing a transdermal or in vitro drug delivery system using a water-soluble polymer material as a matrix. The operation steps of one of the preparation methods are as follows:

According to the proportion of prescriptions, weighed polyvinyl alcohol and added to the appropriate amount of water, 95° C. water bath heating and stirring for 45 min, so that all polyvinyl alcohol dissolved;

According to the prescription ratio, weighed gelatin, sodium carboxymethyl cellulose and added to the solution of step I) in turn, 60-70° C. heating and stirring for 15 min, to make sure the added excipients are completely dissolved and mixed uniformly;

According to the prescription ratio, weighed 70% of plant alcohol, added to the solution of step 2), stirring at 60-70° C.;

According to the prescription ratio, weighed sodium polyacrylate and glycerol, then sodium polyacrylate was added to the glycerol, stirred uniformly, and added to the solution of step 3), 70-80° C. heating 10 min, stirring evenly;

According to the prescription ratio, weigh 0.5 parts of the vegetable oil and the nanobody, add to the solution of step 4), and mix evenly; that is a sustained release system for percutaneous or in vitro administration.

The present invention provides a formulation and a preparation method of a sustained-release transdermal delivery system using a water-soluble polymer material as a matrix, and its preparation and application. The matrix is composed of polyvinyl alcohol, gelatin, sodium carboxymethyl cellulose, plant alcohol, glycerin, sodium polyacrylate, and vegetable oil, mix with water. The drug loading capacity of the matrix is large; it has good affinity with various drugs including plant extracts and chemical drugs; sustained-release drugs have good transdermal effects; breathability and high comfort for skin application; basically no skin irritation and allergic reactions, it is an ideal transdermal delivery platform. In combination with various drugs, it is possible to prepare a variety of sustained-release transdermal preparations.

The application of the percutaneous or in vitro drug delivery system of the present invention is characterized in that it is used for the preparation of a transdermal or in vitro drug formulation of a nanobody drug.

The application of the percutaneous or in vitro drug delivery system of the present invention is characterized in that it is used to prepare and treat autoimmune diseases, in vitro skin and body, such as vagina, oral cavity, nasal cavity, eyes, ear cavity, intestinal near the anus, local inflammation, blood diseases, orthopedic diseases, cancer and other diseases.

The nanobody preparation for percutaneous or in vitro administration of the present invention can be dispersed in water, gel or cream for external application to the skin. It releases active antibodies or antibody fragments on the surface of the skin, allowing active antibodies or antibody fragments to effectively penetrate through the epidermis.

One of the features of the percutaneous or in vitro drug delivery system described in the present invention is that the percutaneous drug delivery preparation for the nanobody of the present invention can effectively penetrate the stratum corneum and eliminate the epidermis. The melanocytes in the basal layer have a good stain treatment and are suitable for the treatment of melasma, age spots, freckles and other pigmentation diseases.

The application of the percutaneous or in vitro drug delivery system according to the present invention is characterized in that, for the percutaneous sustained release drug delivery preparation of the nanobody of the present invention, the drug solution can penetrate deeply into the inner layer of the skin. It can control the antibacterial and anti-inflammatory effects of fats, and can eliminate free radicals and pimples, pustules, and acne, commonly known as acne, on the skin of the face, back of the thoracodorsal and extracorporeal skin, without any side effects on the skin, and can effectively treat facial acne bio-cosmetic liquids and anti-aging cosmetics.

Example #1 Nanobodies in the percutaneous or in vitro delivery system described in the present invention can eliminate inflammatory factors such as IL-1 alpha (Interleukin 1 alpha) TNF-alpha, IL-8 (Interleukin 8).

Example #2 Nanobodies in the percutaneous or in vitro drug delivery systems of the present invention can eliminate skin-infecting bacteria such as Propionibacterium acnes (P. acnes).

Example #3 IL-6 (Interleukin 6) plays an important role in severe inflammatory diseases, creams based on nanobodies tIL-6/IL-6R can be applied to the skin of joints to eliminate autoimmune diseases IL-6 inflammatory factor.

Example #4, IgE target-based nanobody creams may be applied to the throat skin to treat allergic asthma.

Example #5, a cream based on a nanobody that targets a vWF target can be applied to the skin for the treatment of thrombocytopenic purpura (TTP).

Example #6, creams based on ALX-0171-targeted nanobodies can be applied to throat skin treatment for treatment of RSV infection. RSV infection is very common in infants, but there are currently no drugs available.

Example #7, a cream based on RANKL-targeted nanobodies can be applied to the joint skin for the treatment of osteoporosis indications.

Example #8, creams based on anti-tumor nanobodies targeting EGFR, HER2, VEGFR2, c-Met, CXCR7, etc. can be applied to the respective skin and can also form nanoparticles that penetrate in the cancerous area for treatment cancer.

Example #9, creams related to anti-tumor nanobodies can be applied to the corresponding skin and can also have anti-venom and detoxification effects.

Example #10, a cream based on the nanobody Nb An46 can be resistant to infection by African Trypanosoma.

Example #11, creams based on specific inhibition of TNFR1 trivalent nanobodies can be used for anti-inflammatory diseases.

The nanobody of the present invention may be administered to a pharmaceutical preparation percutaneously or in vitro and may contain active microorganisms. The antibody or antibody fragment may be expressed and/or secreted on the surface of the skin.

The nanobody drug formulation according to any one of the above claims of the present invention, wherein the antibody is a VHH type or VNAR type heavy chain immunoglobulin or a fragment thereof, preferably derived from Camelids, most preferably derived from a llama heavy chain antibody or a fragment thereof, or an antibody is an immunoglobulin heavy chain or light chain domain antibody (dAb) or a fragment thereof.

The nanobody of the present invention provides a pharmaceutical preparation percutaneously or in vitro, improves bioavailability, reduces dosage, reduces adverse reactions, increases drug treatment index, and increases clinical drug safety and formulation compliance. Therefore, the percutaneous or in vitro pharmaceutical preparations of the nanobodies of the present invention not only have advantages superior to those of conventional drug delivery systems, but also can achieve multi-pathway transdermal administration. For example, it can be used for oral administration, pulmonary administration, ophthalmic administration, and nasal administration. However, at present in the world, there is no application of the nanobody described in the present invention to a pharmaceutical preparation percutaneously or in vitro.

Some examples of VHH sequences of nanobodies that can be implemented in the present invention are:

VHH-αHER2, Sequence 1: 128 amino acids
DVQLVESGGG, SVQGAAGGSL, RLSCAASDIT, YSTDCMGWFR,
QAPGKEREGV, ATINNGRAIT, YYADSVKGRF, TISQDNAKNT,
VYLQMNSLRP, KDTAIYYCAA, RLRAGYCYPA, DYSMDYWGKG,
TQVTVSSG
VHH-αHER2, SEQ ID NO: 126 amino acids
DVQLEESGGG, SVQTGGSLRL, SCAASGYTYS, SACMGWFRQG,
PGKEREAVAD, VNTGGRRTYY, ADSVKGRFTI, SQDNTKDMRY,
LQMNNLKPED, TATYYCATGP, RRRDYGLGPC, DYNYWGQGTQ,
VTVSSG
VHH-αVEGF: Sequence 1: 132 amino acids
MAQVQLQESG, GGSVQDGGSL, RLSCAASGYA, YDTYYMGWFR,
QAPGKEREWV, AGITSLVSGV, AYYKYYTDSV, KGRFTIFRDD,
DKNTVDLQMN, SLKPEDTAIY, YCAASRSGLR, ARLLRPELYE,
YWGQGTQVTV, SS
VHH-αVEGF: Sequence 2: 129 Amino Acids
MAQVQLQESG, GGSVQAGGSL, RLSCVASGDT, YSSACMGWFR,
QAPGKEREGV, ATICTSTSMR, TRYYADAVKA, RFTISQDNAK,
NTVYLQMNSL, KPEDIAMYYC, ATGHTVGSSW, RDPGAWRYWG,
QGTQVTVSS
VHH-αEGFR: Sequence 1: 138 amino acids
QVQLQESGGG, LVQPGGSLRL, SCAASGRTFS, SYAMGWFRQA,
PGKQREFVAA, IRWSGGYTYY, TDSVKGRFTI, SRDNAKTTVY,
LQMNSLKPED, TAVYYCAATY, LSSDYSRYAL, PQRPLDYDYW,
GQGTQVTVSS, LEHHHHH

The above non-humanized nanobody VHH sequence may replace one or more amino acid residues in the amino acid sequence of its naturally-occurring VHH sequence domain with amino acid residues existing at corresponding positions in the conventional human VH sequence domain.

The above described embodiments merely express one of the embodiments of the present invention, and the description thereof is more specific and detailed, but it should not be understood that the scope of the present invention is limited by the patent. It should be pointed out that for a person of ordinary skill in the art, many variations and improvements can be made without departing from the core content of the present invention: the concept of a transdermal or in vitro drug delivery system of a nanobody drug. It belongs to the protection scope of the present invention. Therefore, the scope of protection of the present invention shall be subject to the appended claims.

1. Preparation method and application of transdermal delivery of nanobody biopharmaceuticals, the system includes specific bioactive nanobodies, nanobodies include humanized and non-humanized antibody forms. The transdermal delivery of nanobodies is different from injection and oral dosage forms. The transdermal delivery system delivers the active ingredient through the skin and has the advantages of no pain, self-administration, and treatment at any time. It is simpler to use than the injection dosage form and has high drug delivery efficiency.

2. The nanobody transdermal delivery formulation system in claim 1, wherein the application comprises a nanobody drug for localized administration of the drug, and a nanobody for penetration of the skin into the blood circulation system to reach a predetermined lesion site.

3. The nanobody biomedical transdermal drug delivery formulation system in claim 1, wherein the nanobody is a specific nanobody directed against different target lesions including, but not limited to, targets for dermatological diseases, targets for inflammation and rheumatism, cancer target, virus and bacteria target, cardiovascular disease target, diabetes target, Alzheimer disease target, brain tumor target.

4. The nanobody biopharmaceutical transdermal drug delivery formulation in claim 1, comprising an active nanobody and an antibody drug carrier that maintains and enhances the stability and tissue penetration of the nanobody, including a water soluble macromolecule biosaccharide matrix polyols, polyvinyl alcohol (PEG), dextran, polyamino acids, glycerol, phospholipids, gelatin, sodium carboxymethylcellulose, water, but not limited thereto. The preparation carrier can stabilize the structure and efficacy of the protein, release the drug through the skin, has good transdermal effect, and has high skin application comfort; almost no skin irritation and allergic reaction.

5. The nanobody biopharmaceutical transdermal drug delivery formulation in claim 1, wherein the system can be combined with various specific nanobodies and other drugs to form a composite transdermal drug delivery formulation.

6. The nanobody biopharmaceutical transdermal drug delivery formulation in claim 1, wherein one of the basic formulations of the nanobody drug delivery carrier is prepared from the following auxiliary materials in parts by weight: 18 parts of water-soluble polymer biosaccharides matrix, 10 parts of polyhydroxyl compound, 5 parts of polyvinyl alcohol (PEG), 10 parts of dextran, 10 parts of polyamino acids, 10 parts of glycerin, 5 parts of phospholipid, 2.5 parts of gelatin, 1 part of sodium carboxymethyl cellulose, mix with water.

7. The in vitro percutaneous drug delivery system for nanobody using a water-soluble polymer material as a substrate carrier according to claim 4, wherein one of the preparation methods is characterized in that the steps of the preparation method are as follows: 1) according to the prescription ratio, add polyvinyl alcohol to the appropriate amount of water, heat and stir in a water bath at 95° C. for 45 minutes to completely dissolve the polyvinyl alcohol; 2) according to the prescription ratio, take one part of lactic acid, polyamino acids, glycerin, phospholipids, gelatin, and sodium carboxymethyl cellulose, add to the solution of step I) in turn, and the mixture is heated and stirred at 60-70° C. for 15 min to completely dissolve the added excipients and mix well; Stir evenly to become a sustained release carrier system of nanobody through the skin.

8. The nanobody biopharmaceutical transdermal drug delivery formulation in claim 1, wherein the active nanobody comprises an active antibody or antibody fragment, including a multitarget nanobody polymeric linker.

9. The nanobody biopharmaceutical transdermal drug delivery formulation in claim 1, wherein the administration site includes human skin and body tissue inside the body, such as the oral cavity, nasal cavity, eyes, ear cavity, vagina, and the intestinal tract near the anus.

10. The nanobody biopharmaceutical transdermal drug delivery formulation in claim 1, comprises water, lipid, gel, cream carrier, for application to the skin surface, or push-into the nasal cavity, vagina, and the intestines near the anus by column, or drip into the mouth, nasal cavity, eyes, ear cavity.

11. The nanobody biopharmaceutical transdermal drug delivery preparation system in claim 1, wherein the preparation system comprises a nanoparticle transdermal sustained-release preparation for effective passage through the stratum corneum and comprehensive elimination of melanin deposition of the epidermal basal layer to eliminate or weaken melasma, age spots, and freckles.

12. The nanobody biopharmaceutical transdermal drug delivery preparation system in claim 1, comprises applying to the skin papules, pustules, acne commonly known as pimples, various skin rashes, treating facial acne, and forming a skin care beauty product.

13. The nanobody biopharmaceutical transdermal drug delivery preparation system in claim 1, comprising active probiotic microorganisms for expression and secretion of active nanobodies, or antibody fragments, or nanobody-linked polymers on the surface of the skin.

14. The nanobody biopharmaceutical transdermal drug delivery preparation system in claim 1, comprising an active nanobody-linked polymer, wherein the nanobody and albumin in the nanobody-linked polymer are combined to extend the drug half life in the antibody drug carrier and the human body.

15. The targets of active nanobody according to claim 3 include, but not limited to, HER2 (human epidermal growth factor receptor 2 or HER2/neu), EGFR (epidermal growth factor receptor), VEGF (vascular endothelial growth factor), EGFa (epidermal growth factor a), FGFb (epidermal growth factor b), interferon IL-6, IL-4, IL-5, IL-9, IL-13, IL-17a, TNFa (Tumor Necrosis Factor a), TNFb (Tumor Necrosis Factor b)


SEQUENCE LISTING (5 sequences)

SEQ ID NO: 1 - protein, 128 aa, Lama glama
DVQLVESGGGSVQGAAGGSLRLSCAASDITYSTDCMGWFRQAPGKEREGVATINNGRAITYYADSVKGRFTISQDNAKNTVYLQMNSLRPKDTAIYYCAARLRAGYCYPADYSMDYWGKGTQVTVSSG

SEQ ID NO: 2 - protein, 126 aa, Lama glama
DVQLEESGGGSVQTGGSLRLSCAASGYTYSSACMGWFRQGPGKEREAVADVNTGGRRTYYADSVKGRFTISQDNTKDMRYLQMNNLKPEDTATYYCATGPRRRDYGLGPCDYNYWGQGTQVTVSSG

SEQ ID NO: 3 - protein, 132 aa, Lama glama
MAQVQLQESGGGSVQDGGSLRLSCAASGYAYDTYYMGWFRQAPGKEREWVAGITSLVSGVAYYKYYTDSVKGRFTIFRDDDKNTVDLQMNSLKPEDTAIYYCAASRSGLRARLLRPELYEYWGQGTQVTVSS

SEQ ID NO: 4 - protein, 129 aa, Lama glama
MAQVQLQESGGGSVQAGGSLRLSCVASGDTYSSACMGWFRQAPGKEREGVATICTSTSMRTRYYADAVKARFTISQDNAKNTVYLQMNSLKPEDIAMYYCATGHTVGSSWRDPGAWRYWGQGTQVTVSS

SEQ ID NO: 5 - protein, 138 aa, Lama glama
QVQLQESGGGLVQPGGSLRLSCAASGRTFSSYAMGWFRQAPGKQREFVAAIRWSGGYTYYTDSVKGRFTISRDNAKTTVYLQMNSLKPEDTAVYYCAATYLSSDYSRYALPQRPLDYDYWGQGTQVTVSSLEHHHHHH